(19) United States
(12) Patent Application Publication
(10) Pub. No.: US 2019/0105624 A1
(43) Pub. Date: Apr. 11, 2019

(54) Method for Producing Material Having Foaming and/or Emulsifying Properties by Reacting Oils and Fats with Lipase in Low Moisture State and Product Thereof

(71) Applicant: Shoichi Kobayashi, Ibaraki (JP)

(72) Inventors: Shoichi Kobayashi, Ibaraki (JP); Yuji Kobayashi, Ibaraki (JP)

(21) Appl. No.: 15/727,664

(22) Filed: Oct. 9, 2017

(51) Int. Cl. B01F 17/00; C12P 7/64; C12P 19/00; A23L 33/10

(52) U.S. Cl. CPC B01F 17/0085; B01F 17/0021; C12P 7/64; A23V 2002/00; C12Y 301/01003; C12Y 302/01004; A23L 33/10; C12P 19/00

(57) ABSTRACT

Disclosed herein is a method for producing a raw material having foam-forming properties and/or emulsion-forming properties by causing a lipase to act on an oil/fat in a low moisture state; and a product thereof. As disclosed, an oil/fat, or an oil/fat and a carbohydrate, is reacted with a lipase in a low moisture state, that is, in a state in which a moisture content relative to a dry weight of the oil/fat is 4 to 400 [d.b.%] (an added quantity of water is 2 to 200 μL relative to 50 mg of the oil/fat), thereby producing a carbohydrate-oil/fat-lipase reaction product of a precursor material having latent foam-forming properties and emulsion-forming properties, and an alkaline component or material is added to the precursor material having latent foam-forming properties and emulsion-forming properties, and the obtained mixture is foamed, emulsified or powdered, thereby producing a product in which the entire reaction product can be used as a food material. Also disclosed is a carbohydrate-oil/fat-lipase reaction product of a precursor material having latent foam-forming properties and emulsion-forming properties can be produced; and a powdered and stabilized carbohydrate-oil/fat-lipase reaction product having foam-forming properties and/or emulsion-forming properties, and a product in which the entire reaction product can be used as a food material.

### BACKGROUND OF THE INVENTION

### Field of the Invention

The present invention relates to a method for producing an oil/fat-lipase reaction product or carbohydrate-oil/fat-lipase reaction product of a precursor material having latent foam-forming properties and emulsion-forming properties by mixing an “oil/fat raw material” or a combination of two or more types thereof with a “carbohydrate raw material” and carrying out a lipase reaction in a low moisture state, that is, in a state in which a moisture content relative to a dry weight of the oil/fat is 4 to 400 [d.b.%] (an added quantity of water is 2 to 200 μL relative to 50 mg of the oil/fat) in the presence of a lipase, adding an alkaline component or raw material to these precursor materials so as to produce a material having foam-forming properties and emulsion-forming properties, and using the entire reaction product as a food material, and the present invention more specifically relates to a method for causing an oil/fat-related enzyme lipase to act on an oil/fat raw material in a low moisture state, simultaneously causing a variety of carbohydrate raw materials to act, adding an alkaline component or raw material so as to produce a material having foam-forming properties and emulsion-forming properties, and using the entire reaction product as a highly stable food material either without further modification or after being dried or formed into a paste or powder. In the present specification, “oil/fat raw material” means an oil/fat, and “carbohydrate raw material” means a carbohydrate.

### Description of the Related Art

There are many types of lipase, which are involved in a great number of reactions, and there are therefore many examples of scientific research, practical developments and practical applications involving lipases. The present invention indicates that by carrying out diligent research into methods for facilitating a lipase reaction under specific reaction conditions, altering the characteristics of an oil/fat and producing a material having a broader scope of use, it is possible to inexpensively produce and provide a safe and novel material. As a result of investigating related prior art, products similar to the present invention were not found among inventions other than inventions of the inventors of the present invention. In addition, bread, fermented foods, and the like, were produced in the past in some cases by reducing the moisture content, and a variety of enzymes were involved, but it is not possible to specify clear conditions such as those used in the present invention in reactions in such mixed systems. The method of the present invention can be combined with some conventional production methods.

Among the prior art documents, Non-patent reference 1 reports a variety of reaction methods involving lipases, and this document indicates that lipases are involved in hydrolysis, transesterification, esterification, alcoholysis, acid cleavage and aminolysis reactions, act on a variety of substrates including oils/fats, triglycerides and esters, exhibit resistance to solvents, and can be used in a broad range of applications, indicates that transesterification, hydrolysis and esterification are particularly useful in food processing and the like, and discloses an extremely large number of practical examples as relevant prior art.

Non-patent reference 2 reports a method for synthesizing a glyceride by agitating and reacting ethyl caprate and glycerol (having a moisture content of 4%) at a temperature of 40° C. using a lipase formulation immobilized on a polypropylene material. However, documents relating to “lipase reactions in a low moisture state” cannot be found among the prior art documents, and several documents have been found as related documents, such as Non-patent reference 2, which relates to synthesis of a variety of materials/components by means of lipase reactions “in which organic solvents are not used”. Many other methods involving reactions in a low moisture state have been proposed.

Non-patent reference 3 reports a method for synthesizing a wax by mixing a long chain alcohol (stearyl alcohol) and a methyl ester (methyl oleate), and carrying out a transesterification reaction (an alcohol exchange reaction) using an immobilized lipase (Lipozyme IM). However, a solvent is not added in this reaction, and the reaction is carried out under specific conditions, namely an enzyme concentration of 0.12% to 2% and a temperature of 55° C. to 65° C.

Non-patent reference 4 reports a method of adding 10% of water to a solid reaction substrate mixture and carrying out a reaction using Thermoase PS 160 as a “solid-to-solid synthesis”, and explains that this reaction progresses in a saturated substrate liquid phase, with the reaction product precipitating.

Non-patent reference 5 reports a method for synthesizing a glucose-fatty acid monoester by mixing glucose with a fatty acid or fatty acid methyl ester and carrying out a reaction using a polypropylene EP 100-immobilized lipase. This document explains that by carrying out a reaction under reduced pressure at a temperature of 60° C. in ethyl methyl ketone or acetone, the yield is 90% or more, that the yield is increased by the solvent vaporizing, and that the glucose-fatty acid monoester can be readily used as a food material.

Non-patent reference 6 reports a method for producing a fatty acid-modified lipase by mixing an aqueous lipase solution with an ethanol solution in which a fatty acid is dissolved, carrying out centrifugal separation, and freeze-drying the obtained sediment, and this document discloses carrying out a transesterification reaction between triolein and palmitic acid in hexane using this fatty acid-modified lipase, and evaluating the activity thereof. However, this method is characterized by a reaction involving the use of a fatty acid-modified lipase.

Non-patent reference 7 reports a method of mixing 4-methyloctanoic acid and a variety of polyethylene glycol compounds and converting to mono-esters or di-esters at a variety of water activities using Novozym 435, which is a lipase immobilized on an acrylic resin. However, this method is characterized by a reaction involving the use of an immobilized lipase.

In addition, Non-patent reference 8 reports a method relating to “an enzyme reaction in which an ionic liquid is used as a medium”, and Non-patent reference 9 reports a method relating to “synthesis of a useful substance by means of an enzyme reaction in supercritical CO₂”, but these methods focus on enzyme reactions involving special methods and are attracting attention for research and development, but it is difficult to imagine these methods being put to practical use at the present time.

Next, Patent reference 1 proposes a method “for producing a fatty acid ester by carrying out a transesterification reaction or esterification reaction using a chemically modified lipase in an O/W emulsion comprising an oil/fat or fatty acid ester, an alcohol and water or in an O/W emulsion comprising a fatty acid, an alcohol and water”. However, this method differs from the present invention by specifying use of a chemically modified lipase as an essential constituent feature.

As a feature relating to an enzyme reaction in a low moisture state that the inventors of the present invention have developed and applied for a patent, Patent reference 2 and food material produced using the method” proposes “a method for producing a food material, the method being characterized by mixing grain flour or starch with an oil/fat, reacting a lipase with the grain flour or starch in a pseudo-powdery state having a prescribed moisture content and oil/fat content relative to the grain flour or starch so as to hydrolyze the oil/fat and produce a food material constituted from the reaction mixture”.

Patent reference 3 proposes “a method of causing a lipase to act and using starch granules or a cellulose powder as a carrier”. However, it is explicitly indicated that this method “can also be used in a lipase reaction, for example, can be used to produce a fatty acid ester by spray mixing a fatty acid and a sugar alcohol as base materials on a powdered carrier and carrying out a sealed static reaction at a temperature of 50° C. to 60° C.”, and the purpose of the invention is to produce a fatty acid ester by mixing a fatty acid and sugar alcohol.

Patent reference 4 proposes “a method for producing a fatty acid ester”, and explicitly indicates that this method “can also be used in a lipase reaction, for example, can be used to produce a fatty acid ester by spray mixing a fatty acid and a sugar alcohol as base materials on a powdered carrier and carrying out a sealed static reaction at a temperature of 50° C. to 60° C.”, and matters disclosed in this document are the same as those disclosed in Patent reference 3. Therefore, according to prior art, use of methods involving use of modified lipases is prevalent in cases where a reverse reaction of a lipase is used, and it was extremely difficult in the past to produce a variety of reaction products by carrying out reverse reactions of lipases by means of simple methods and conditions using conventional lipases.

Patent reference 5 proposes “a method for producing an oil/fat-alcohol complex composition by a reverse reaction of a lipase in a pseudo-powder state; and a food material comprising the complex composition”. However, this method is a method comprising subjecting an alcohol (including a compound having a lipase reverse reaction-capable OH group in the molecule) and an oil/fat to a lipase reverse reaction in a pseudo-powder state, and differs from the present invention in terms of raw material composition.

Paragraph [0001] in this document indicates that “the present invention relates to a method for producing an oil/fat-alcohol complex composition by agitating and mixing a powder base material with an oil/fat raw material (excluding DHA- and EPA-related materials) and an alcohol raw material as reaction substrates, and carrying out a lipase reverse reaction in a pseudo-powder state having a dry basis moisture content of 20 to 200 [d.b.%] in the presence of an oil/fat-related enzyme lipase so as to enable use of the entire reaction product as a food material; and a food material comprising the complex composition, and an enzyme reaction in a pseudo-powder state is known as a para-powder state enzyme reaction (PPSER)”, and paragraph [0002] in this document, the weight of the powder base material is denoted by Wd, the weight of a liquid substance such as water or an oil/fat is denoted by Ww, and the value of Md=Ww×100/Wd is defined as the dry basis moisture content [d.b.%]”, and it is therefore clear that the method disclosed in this document is fundamentally different from the method of the present invention.

Patent reference 6 proposes “a method for producing an oil/fat-menthols, cholesterols, or polyphenols complex composition by a reverse reaction of a lipase in a pseudo-powder state; and a food material comprising the complex composition”. However, this method is characterized by specifying the type of alcohol in the method disclosed in the Patent reference 6.

### PATENT REFERENCE

[Patent reference 1] Japanese Patent Application Publication No. 2006-50954: “Method for producing fatty acid ester using modified lipase”
[Patent reference 2] Japanese Patent Application No. 2015-107097A: “Method for producing food material having emulsification ability”
[Patent reference 3] Japanese Patent No. 5282252: “Starch granule or cellulose powder immobilized lipase, and method for producing fat or fatty oil reaction product”
[Patent reference 4] Japanese Patent Application Publication No. 2009-060875: “Method for producing functional food material by mixing carbohydrate and other food ingredient together followed by high-temperature treatment in air, and the resultant functional food material”
[Patent reference 5] Japanese Patent Application No. 2014-199272: “Method for producing oils and fats-alcohols complex composition by performing reverse reaction of lipase in pseudo-powder state, and food material composed of complex composition”
[Patent reference 6] Japanese Patent Application No. 2014-199422: “Method for producing oils and fats-menthols, cholesterols, or polyphenols complex composition by performing reverse reaction of lipase in pseudo-powder state, and food material composed of complex composition”

### NON-PATENT REFERENCE

[Non-patent reference 1] R Aravindan et al., “Lipase applications in food industry”, Indian Journal of Biotechnology, Vol 6, pp 141-158 (2007)
[Non-patent reference 2] Anna Millqvist Fureby et al., “Glyceride synthesis in a solvent-free system”, Journal of the American Oil Chemists' Society, Volume 73, Issue 11, pp 1489-1495 (1996)
[Non-patent reference 3] N. Goma-Doncescu and M. D. Legoy, “An original transesterification route for fatty acid ester production from vegetable oils in a solvent-free system”, Journal of the American Oil Chemists' Society, Volume 74, Issue 9, pp 1137-1143 (1997)
[Non-patent reference 4] Markus Erbeldinger et al., “Scale-up of Enzymatic Solid-to-Solid Peptide Synthesis and Enzyme Recovery”, AIChE Journal, Vol. 47, No. 2, pp 500-508 (2001)
[Non-patent reference 5] Youchun Yan et al., “Lipase-catalyzed solid-phase synthesis of sugar fatty acid esters: Removal of byproducts by azeotropic distillation”, Enzyme and Microbial Technology, Volume 25, Issues 8-9, pp 725-728 (1999)
[Non-patent reference 6] Tatsuo MARUYAMA, “Lipase activation for modification of fats and oils”, University of Tokyo dissertation, 29 Mar. 2002
[Non-patent reference 7] N. W. J. T. Heinsman et al., “ESTERIFICATION OF 4-METHYLOCTANOIC ACID WITH POLYETHYLENE GLYCOL AT DIFFERENT aw”, Biocatalysis and Biotransformation, 2001, Vol. 19, No. 3, pp 181-189 (2001)
[Non-patent reference 8] Toshiyuki ITO, “Development of enzyme reaction using ionic liquid as medium”, Grant-in-Aid for Scientific Research Report, 20 May 2010
[Non-patent reference 9] Tomoko MATSUDA, “Organic synthesis by enzymes in supercritical CO₂”, The Chemical Times, No. 3 (197), pp 8-13 (2005)

### SUMMARY OF THE INVENTION

Use of liquid phase reactions is commonplace when producing processed/chemical products by means of enzyme reactions, and products are produced by completing a reaction, taking out the product and then concentrating or drying the product without further modification, and even in cases where immobilized enzymes are used, products are produced by passing a liquid. In this type of production method, however, large quantities of water are required, and in order to obtain a highly stable dried product, the product must be concentrated or dried, and the resulting increase in production costs and increase in environmental burden have an effect on product price. There are examples in which it is possible to obtain a liquid product by carrying out concentration only, but obtaining a dried powder is advantageous from the perspectives of maintaining quality, storage and handling.

In general, it is thought that enzyme reactions progress in a powdered state, and the moisture content in a material obtained by adding an enzyme to a mixture comprising equal quantities of a reaction raw material and a carbohydrate used to maintain a powdered state in a carbohydrate-related enzyme is 14% to 50% in the case of starch or grain flour, and even in the case of a lipase reaction, it is thought that an enzyme reaction will progress at a moisture content within the range of 20% to 50%. However, incases where a useful product is produced using a lipase reaction, it is thought that it is essential to specify conditions for facilitating the lipase reaction and conditions for selecting/combining an oil/fat and/or other materials.

### Subject to be Solved by the Invention

Under such circumstances and with the prior art mentioned above in mind, the inventors of the present invention carried out diligent research into producing a useful material by means of a lipase reaction, thereby succeeding in finding that it is possible to achieve the desired objective by specifying conditions for facilitating a lipase reaction and conditions for selecting/combining an oil/fat and/or a carbohydrate component/material, and completing the present invention.

The purpose of the present invention is to provide a method for producing a precursor material having latent foam-forming properties and emulsion-forming properties by adding a carbohydrate to an oil/fat or a combination of two or more types thereof and causing a lipase to act on the oil/fat in a low moisture state; a method for producing a novel material having foam-forming properties and emulsion-forming properties by mixing an oil/fat with an alkaline component or material and facilitating a lipase reaction in a low moisture state; and materials comprising these reaction products. Having foam-forming properties means exhibiting an interfacial activation effect, and in cases where foam-forming properties are not required, mild agitation or a defoaming treatment should be conducted without agitating by shaking, and the purpose of the present invention is to provide both of these materials.

### Means for Solving the Subject

In order to solve the problem mentioned above, the present invention is configured from the following technical means.
(1) A method for producing an oil/fat-lipase reaction product of a precursor material having latent foam-forming properties and emulsion-forming properties by reacting an oil/fat or a combination of two or more types thereof with an enzyme lipase in a low moisture state in the presence of a lipase, the method comprising carrying out a lipase reaction in which a moisture content relative to a dry weight of the oil/fat is 4 to 400 [d.b.%] (an added quantity of water is 2 to 200 μL relative to 50 mg of the oil/fat) as the enzyme reaction in a low moisture state.
(2) A method for producing a carbohydrate-oil/fat-lipase reaction product of a precursor material having latent foam-forming properties and emulsion-forming properties by adding a carbohydrate to an oil/fat or a combination of two or more types thereof and carrying out an enzyme reaction in a low moisture state in the presence of a lipase,
   the method comprising carrying out a lipase reaction in which a moisture content relative to a dry weight of the oil/fat is 4 to 400 [d.b.%] (an added quantity of water is 2 to 200 μL relative to 50 mg of the oil/fat) as the enzyme reaction in a low moisture state.
(3) A method for producing an oil/fat-lipase reaction product or carbohydrate-oil/fat-lipase reaction product having latent foam-forming properties and emulsion-forming properties, the method comprising adding an alkaline component or raw material to the oil/fat-lipase reaction product or carbohydrate-oil/fat-lipase reaction product of a precursor material having latent foam-forming properties and emulsion-forming properties obtained in above (1) or (2), and foaming, emulsifying or powdering, thereby using the entire reaction product as a food material.
(4) The method according to above (1) or (2), wherein rapeseed oil, soy bean oil, palm oil, coconut oil, cooking oil, Resetta, corn oil, safflower oil, olive oil, sesame oil, sunflower oil, rice oil or linseed oil, which are plant-based oils/fats; fish oil, whale oil, horse oil, lard or butter, which are animal-based oils/fats; or DHA, EPA, arachidonic acid, oleic acid, linolic acid or linolenic acid, which are fatty acids, is used as the oil/fat.
(5) The method according to above (2), wherein xylose, fructose, acetylglucosamine, glucose, maltose, soluble starch, sorbitol, erythritol, xylitol, mannitol, sucrose, lactose, trehalose, corn starch, rice starch, rice flour (top-grade rice flour) or cellulose is used as the carbohydrate.
(6) The method according to above (3), wherein sodium hydroxide, potassium hydroxide, sodium carbonate, sodium hydrogen carbonate, potassium carbonate, potassium hydrogen carbonate, sodium phosphate, sodium hydrogen phosphate, soda ash, calcium carbonate (seashell calcium), CalMag-S, calcium phosphate (Fired Bonical) or an ash extraction liquid is used as the alkaline component or raw material.
(7) The method according to above (2), wherein grain flour or starch is used as the carbohydrate and an α-amylase and/or a glucosidase is also used.
(8) The method according to above (7), wherein the grain flour is rice flour, white rice bran or wheat flour while the starch is waxy potato starch, sweet potato starch, tapioca starch, non-glutinous rice starch, glutinous rice starch, corn starch, glutinous corn starch, wheat starch or sago starch.
(9) The method according to above (5), wherein cellulose is used as the carbohydrate and a cellulase is also used.
(10) The method according to above (3), wherein an ash extraction liquid is added as an alkaline material.

The present invention will now be explained in greater detail.

The present invention is a method for producing an oil/fat-lipase reaction product of a precursor material having latent foam-forming properties and emulsion-forming properties by reacting an oil/fat or a combination of two or more types thereof with a lipase in a low moisture state, that is, in a state whereby the moisture content relative to the dry weight of the oil/fat is 4 to 400 [d.b.%] (the added quantity of water is 2 to 200 μL relative to 50 mg of the oil/fat), in the presence of a lipase, and producing an oil/fat-lipase reaction product having latent foam-forming properties and emulsion-forming properties by adding an alkaline component or material to the oil/fat-lipase reaction product of a precursor material, wherein by carrying out enzyme deactivation and sterilization by treating the reaction product for approximately 30 minutes at a temperature of 105° C., the entire reaction product can be used as a food material.

Examples of oils/fats include oils/fats that are solid at room temperature, such as lard and butter, and oils/fats that are in a sol-gel state, such as palm oil, but when these oils/fats are mixed with liquid oils/fats such as rapeseed oil or rice oil after being melted through heating, and then shaken in a boiling water bath, the oils/fats dissolve and become easy to handle. The method of the present invention can also be used for combinations of plant-based oils/fats and animal-based oils/fats such as lard and beef tallow. In addition, by devising a concentrating method or the like, use may be possible at a moisture content relative to the dry weight of an oil/fat that falls outside the scope of the present invention.

By adding an alkaline component or material to a reaction product produced in this way, it is possible to obtain a material having latent foam-forming properties and emulsion-forming properties (a product having latent functionality). Therefore, by adding an alkaline component or material to this oil/fat-lipase reaction product, it is possible to obtain a material having foam-forming properties and emulsion-forming properties (an oil/fat-lipase reaction product or carbohydrate-oil/fat-lipase reaction product), and these entire reaction products can be used as food materials.

The added quantity of the alkaline component or material is not limited, and foam-forming properties and emulsion-forming properties are achieved even when the added quantity of the alkaline component or material is low. From the perspective of product stability, achieving an optimal pH is desirable, and it is also desirable to know the optimal added quantity. In such cases, the type of component or material is not limited as long as the component or material is alkaline, and it is possible to use, for example, ammonia, amine or carbonate or phosphate of an alkali metal, or the like. An ash extraction liquid can be one obtained by, for example, obtaining a dispersion liquid containing approximately 15% (w/v) of ash obtained by burning rice husks, rice straw, straw, sawdust or pruned wood chips, vigorously agitating by shaking, and then carrying out centrifugal separation. In the present invention, ash obtained by combusting pruned wood chips from plum trees can be advantageously used.

In the present invention, a variety of materials can be produced by using a carbohydrate when producing the oil/fat-lipase reaction product. There are many types of carbohydrate, of which characteristics are varied, and the type of carbohydrate to be used in the present invention is not limited, but maltooligosaccharides such as sucrose and maltose, sugar alcohols such as sorbitol and polysaccharides achieve the effect of powdering and stabilizing a product. In addition, polysaccharides can be used in reactions together with hydrolases thereof. In particular, cellulose is partially solubilized/dispersed by the action of a lipase, and this solubilization/dispersion is further facilitated by additionally using a cellulase, and it is possible to use two or more types of carbohydrate.

To explain enzymes used in the present invention in greater detail, there are an extremely large number of lipase-based enzymes in academic terms, of which EC numbers (Enzyme Commission Numbers) are classified as [EC 3 Hydrolases, EC 3.1 Acting on Ester Bonds, EC 3.1.1 Carboxylic Ester Hydrolases, EC 3.1.1.3 triacylglycerol lipase] or the like, and triacylglycerol lipase is found in a variety of microorganisms and the like, the number of which is extremely large (http://www.brenda-enzymes.info/php/result_flat.php4?ecno=3.1.1.3).

The “List of items included in the existing additive list” includes No.: 305, Name: Phospholipase, Item Name/Other Name: Phosphatidase lecithinase, Origin⋅Production Method⋅Nature: Obtained by extraction with cold to room temperature water from the pancreas of animals or from Brassica oleracea LINNE, or obtained by extraction with cold to room temperature water from a culture medium of filamentous bacteria (Aspergillus oryzae, Aspergillus niger), basidiomycetes (Corticium), actinomycetes (Actinomadura, Nocardiopsis) or bacteria (Bacillus), removing bacteria, concentrating at a cold temperature or room temperature or treating the above with hydrous ethanol or hydrous acetone, carrying out resin purification, and then treating with an aqueous alkaline solution.

This list also includes No. 349, Name: Lipase, Item Name/Other Name: Lipolytic enzyme, Simple Name or Classification Name: Esterase, Origin⋅Production Method⋅Nature: Obtained by extraction with cold to lukewarm temperature water from the internal organs of animals and fish or the sublingual region of animals, or obtained by extraction with cold to lukewarm temperature water from a culture medium of filamentous bacteria (Aspergillus awamori, Aspergillus niger, Aspergillus oryzae, Aspergillus phoenicis, Aspergillus usamii, Geotrichum candidum, Humicola, Mucor javanicus, Mucor miehei, Penicillium camembertii, Penicillium chrysogenum, Penicillum roquefortii, Rhizomucor miehei, Rhizopus delemar, Rhizopus japonicus, Rhizopus miehei, Rhizopus niveus, Rhizopus oryzae), actinomycetes (Streptomyces), bacteria (Alcaligenes, Arthrobactor, Chromobacterium viscosum, Pseudomonas, Serratia marcescens) or yeast (Candida), removing bacteria and then concentrating at a cold temperature or room temperature or treating with ethanol, hydrous ethanol or acetone.

For commercially available products, see the results of a questionnaire carried out in June 2011 by Enzyme Research Unit of Food Research Institute of National Agriculture and Food Research Organization (an independent administrative institution) (http://www.nfri.affrc.go.jp/yakudachi/koso/3_shishitu3_1.index.html).

Enzyme preparations able to be used as the lipase in the present invention can be ordinary lipases, for example lipases produced in 1998, 2010 or 2012 by Amano Pharmaceutical Co., Ltd. (now Amano Enzyme Inc.), such as L1 to L11 below, and the inventors of the present invention have confirmed that these lipases achieve a similar reaction effect despite being somewhat different from each other. In the method of the present invention, L8: Lipase AY “Amano” 30SD, 2012, which exhibited the highest effect among the enzyme agents listed below, was mainly used, and a 1% distilled water solution of a powdered enzyme material of this lipase was used. Hereinafter, this enzyme liquid is abbreviated to LE.
L1: Lipase F-AP15, 1998 (symbol; enzyme symbols, year of procurement, composition, etc.)
L2: Lipase M “Amano” 10, 1998
L3: Lipase A “Amano” 6, 2010, lipase: 48.0%, food material: 52.0%
L4: Lipase AY “Amano” 30G, 2010, lipase: 20.0%, guar gum: 0.4%, food material: 79.6%
L5: Lipase R “Amano”, 2010, lipase: 20.0%, food material: 80.0% (some of the raw materials contain gelatin)
L6: Lipase G “Amano” 50, 2010, lipase: 50.0%, food material: 50.0% (some of the raw materials contain gelatin)
L7: Lipase AY “Amano” 30, 1998
L8: Lipase AY “Amano” 30SD, 2012
L9: Lipase GW “Amano” 50, 2012
L10: Lipase DF “Amano” 15, 2012
L11: Newlase F, 2012

The following three types of lipase produced by Nagase ChemteX Corporation were also tested.
PLA2 Nagase 10P/R
Denabake RICH
PLA2 Nagase L/R

In the present invention, causing a lipase to act on an oil/fat in a low moisture state means carrying out a lipase reaction in a low moisture state, that is, in a state whereby the moisture content relative to the dry weight of the oil/fat is 4 to 400 [d.b.%] (the added quantity of water is 2 to 200 μL relative to 50 mg of the oil/fat). In the present invention, “low moisture state” is a state specified by a specific numerical range, namely a moisture content relative to the dry weight of the oil/fat of 4 to 400 [d.b.%], and in terms of the quantity of water added to the oil/fat, more specifically means that the quantity of water added to 50 mg of the oil/fat is 2 to 200 μL (water in terms of μL units/oil/fat in terms of mg units). In the method of the present invention, the reaction is loosely translated into English in enzyme reaction terms as a Low Moisture State Enzyme Reaction, with this reaction being abbreviated to LMER, and in the present invention, this low moisture state enzyme reaction is referred to as a “low moisture enzyme reaction”, and the terms “LMER method” and “LMER material” are also used. In the present invention, low moisture is sometimes referred to as microaqueous, the terms low moisture reaction, low moisture treatment, microaqueous reaction and microaqueous treatment are sometimes used, and the pseudo-powder reaction disclosed in Patent reference 5 is an example of a related special reaction.

In the present specification, a product produced using an oil/fat, water and a lipase as raw materials is an oil/fat-lipase reaction product of a precursor material (also abbreviated to an oil/fat low moisture reaction precursor material or oil/fat LMER precursor material), and a product produced by adding a carbohydrate to the raw materials mentioned above is a carbohydrate-oil/fat-lipase reaction product of a precursor material (also abbreviated to a carbohydrate-oil/fat low moisture reaction precursor material or carbohydrate-oil/fat LMER precursor material). These precursor materials are materials that exhibit latent foam-forming properties and emulsion-forming properties without the addition of an alkaline component or material, and by adding an alkaline component or material to these precursor materials, it is possible to produce an oil/fat-lipase reaction product (also abbreviated to an oil/fat low moisture reaction or oil/fat LMER material) or carbohydrate-oil/fat-lipase reaction product (also abbreviated to a carbohydrate-oil/fat low moisture reaction or carbohydrate-oil/fat LMER material) having foam-forming properties and emulsion-forming properties.

In the present invention, the weight of the oil/fat is denoted by Wd, the weight of water is denoted by Ww, and the value of Md=Ww×100/Wd is defined as the content relative to the dry weight of the oil/fat [d.b.%]. The amount of carbohydrate to be added/mixed is arbitrary, and does not affect the value of Md. In the present invention, “enzyme reaction in a low moisture state (low moisture enzyme reaction)” means a lipase reaction carried out in a low moisture state, that is, in a state whereby the moisture content relative to the dry weight of the oil/fat is 4 to 400 [d.b.%] (the added quantity of water is 2 to 200 μL relative to 50 mg of the oil/fat). As explanations for terms used in the present specification, “foaming; foam-forming properties” means a characteristic determined from the quantity of foam generated when the preparation of the present invention is added to an aqueous solution and mixed by being agitated, “emulsifying; emulsion-forming properties” means the characteristic of forming a dispersion liquid comprising liquids that do not mix with each other (are not compatible with each other) in an oil-in-water (O/W) type or water-in-oil (W/O) type emulsion, and in the present specification, emulsion-forming properties are sometimes referred to as “emulsion-forming properties/homogeneous dispersibility”.

In terms of test classifications, an untested (untreated) group is abbreviated to “Bnk”, a control test group is abbreviated to “Ref” and a test group is abbreviated to “Exp”. In addition, reagents used in tests in the present invention are special grade reagents, unless explicitly stated otherwise. Commercially available food materials were used for powder base materials and oils/fats. In terms of reaction times, h denotes “hours” and d denotes “days”. Abbreviations for carbohydrates are as follows. Glucose: Glc, maltose: G2, sucrose: Suc, and abbreviations for oils/fats are as follows. Oleic acid: OLA, eicosapentaenoic acid: EPA, docosahexaenoic acid: DHA. Lipase is abbreviated to LE.

The pH was checked using a pH test paper (TEST PAPER ADVANTEC GRADE UNIV pH 1-11). In addition, absorbance was measured using a HITACHI U-2900 UV/VIS Spectrophotometer by adding/mixing 200 μL of a reaction liquid in an absorbance measurement cell containing 2 mL of water, and absorbance is shown as 11 times the measured value.

### Effect of the Invention

Particular effects such as those described below are achieved by the present invention.
(1) It is possible to produce an oil/fat-lipase reaction product of a precursor material having latent foam-forming properties and emulsion-forming properties by causing a lipase to act on a variety of oils/fats, or combinations of two or more types thereof, in a low moisture state.
(2) It is possible to produce a carbohydrate-oil/fat-lipase reaction product of a precursor material having latent foam-forming properties and emulsion-forming properties by adding a carbohydrate to a variety of oils/fats, or combinations of two or more types thereof, and then causing a lipase to act on the obtained mixture in a low moisture state.
(3) By adding an alkaline component or material to the oil/fat-lipase reaction product of a precursor material or carbohydrate-oil/fat-lipase reaction product of a precursor material having latent foam-forming properties and emulsion-forming properties, and then foaming, emulsifying or powdering, the entire reaction product can be used as a food material.
(4) By using rice flour, white rice bran, wheat flour or starch as a carbohydrate, additionally using an α-amylase and/or a glucosidase, and adding an alkaline component or material, it is possible to produce a carbohydrate-oil/fat-lipase reaction product material having foam-forming properties and emulsion-forming properties.
(5) By using cellulose as a carbohydrate, additionally using a cellulase, and adding an alkaline component or material, it is possible to produce a carbohydrate-oil/fat-lipase reaction product material having foam-forming properties and emulsion-forming properties.
(6) It is possible to provide a food material comprising an oil/fat-lipase reaction product having foam-forming properties and/or emulsion-forming properties.
(7) It is possible to provide a food material comprising a carbohydrate-oil/fat-lipase reaction product having foam-forming properties and/or emulsion-forming properties.
(8) By selecting the type of oil/fat and carbohydrate, it is possible to provide a food material having foam-forming properties and/or emulsion-forming properties and having optional specific characteristics.

### BRIEF DESCRIPTION OF THE DRAWINGS

FIG. 1A shows the appearance of reaction systems in which the added quantity of water was altered, and FIG. 1B shows the appearance after adding water and an alkali and then agitating by shaking;

FIG. 2 shows fluctuations in foam-forming properties and emulsion-forming properties as a result of changes in the added quantity of water;

FIG. 3A shows foam-forming properties in systems to which active enzymes were added, FIG. 3B shows foam-forming properties in systems to which deactivated enzymes were added, and FIG. 3C shows foam-forming properties brought about by subjecting a variety of oils/fats to alkali treatment;

FIG. 4A shows systems to which active enzymes were added, and FIG. 4B shows systems to which deactivated enzymes were added;

FIG. 5A shows systems to which active enzymes were added, and FIG. 5B shows systems to which deactivated enzymes were added;

FIG. 6A shows systems to which active enzymes were added (and to which alkalis were not added), FIG. 6B shows systems to which active enzymes were added, and FIG. 6C shows systems to which deactivated enzymes were added;

FIG. 7 shows degrees of coloration (the upper photograph shows systems to which active enzymes were added, and the lower photograph shows systems to which deactivated enzymes were added); and

FIG. 8A shows changes in foam-forming properties and emulsion-forming properties according to the added quantity of alkali, and FIG. 8B shows foam-forming properties and emulsion-forming properties brought about by adding a variety of alkaline materials and components.

### DESCRIPTION OF THE PREFERRED EMBODIMENTS

The present invention will now be explained in greater detail through the use of test examples. Distilled water was used as water in the test examples described below. Commercially available products were used without modification as the raw material oils/fats, fatty acids and carbohydrates.

### Test Example 1

In this test example, a variety of changes [fluctuations in the amount of sucrose+coconut oil+water] brought about by the amount of water in the low moisture state enzyme reaction (LMER) of the present invention were investigated.

50 mg of coconut oil as an oil/fat and 0 to 5 mL of water were added and tested. Specifically, 0 μL, 10 μL, 20 μL, 50 μL, 100 μL, 200 μL, 500 μL, 1 mL or 5 mL of water was added to a mixture comprising 50 mg of coconut oil and 250 mg of sucrose (Suc), and the overall reaction system was observed.

Moreover, in cases where the added quantity of water was 0 μL, 10 μL or 20 μL, approximately 0.5 mg of a powdered lipase enzyme was placed in a vial and 0 μL, 10 μL or 20 μL of water was added. In tests where the added quantity of water was 50 μL or more, a test group was obtained by dispensing 50 μL of a lipase solution having a concentration of 1% (obtained by dissolving a lipase in distilled water) and adding 0 μL, 50 μL, 150 μL . . . of water to the solution.

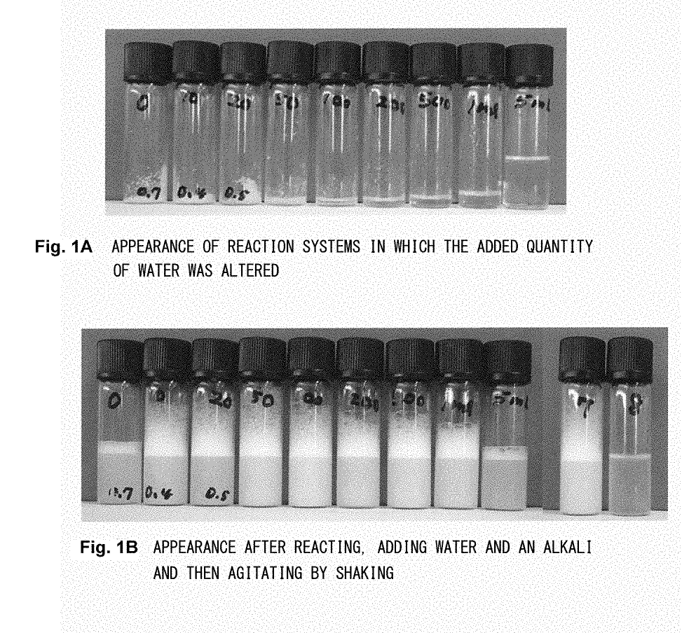

The appearance of a test liquid was observed and compared after reacting for 3 hours at a temperature of 40° C. in a closed system, adding 5 mL, 4.99 mL . . . 4 mL, 0 mL of water so as to make the overall volume of the test liquid up to 5 mL (at this point, the pH was approximately 7 in every test group), agitating by shaking, adding 170 μL of 1N NaOH (at this point, the pH was approximately 9 in those test groups in which the added quantity of water was 20 μL, 50 μL and 100 μL, and 11 or higher in other test groups), agitating by shaking and then immediately allowing the test liquid to stand for 10 minutes. These results are shown in FIGS. 1A and 1B.

FIG. 1A shows the appearance of reaction systems in which the added quantity of water was altered, and FIG. 1B shows the appearance after making the total quantity of water 5 mL following the reaction, adding 170 μL of 1N NaOH, agitating by shaking, and then allowing to stand for 10 minutes.

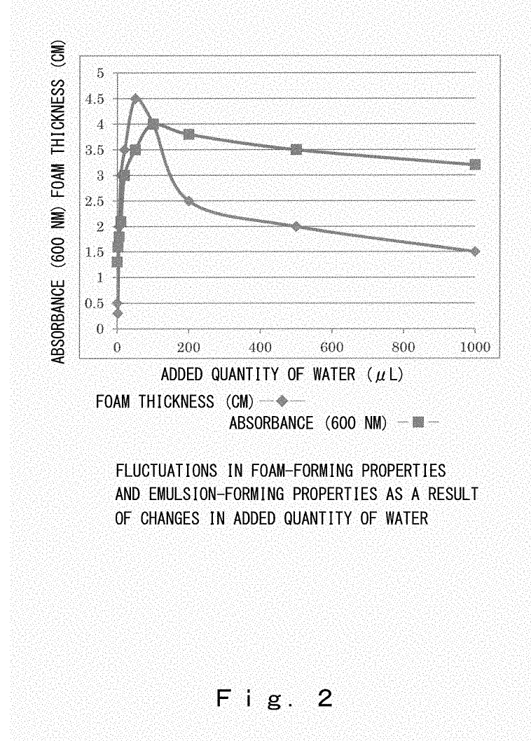

FIG. 2 shows fluctuations in foam-forming properties and emulsion-forming properties as a result of changes in the added quantity of water. Here, foam-forming properties exhibited according to the added quantity of water (μL) were determined by showing values obtained by measuring the thickness (cm) of foam in the space in the vial on the graph and emulsion-forming properties exhibited according to the added quantity of water (μL) were determined by showing values obtained by measuring homogeneous turbidity at a wavelength of 600 nm on the graph.

Foam thickness and absorbance values for Ref and Bnk were 4.0 and 3.3, and 0 and 0.7, respectively.

Moreover, 7 in FIG. 1B is for Ref: 250 mg of Suc+50 mg of coconut oil+50 μL of LE, and shows the appearance after reacting for 3 hours at a temperature of 40° C. in a closed system, carrying out a heat treatment for 10 minutes in a boiling water bath, adding 5 mL of water, adding 170 μL of 1N NaOH, agitating by shaking, and then allowing to stand for 10 minutes. 8 In FIG. 1B is for Bnk: 250 mg of Suc+50 mg of coconut oil+50 μL of deactivated LE, and shows the appearance after reacting for 3 hours at a temperature of 40° C. in a closed system, carrying out a heat treatment for 10 minutes in a boiling water bath, adding 5 mL of water, adding 170 μL of 1N NaOH, agitating by shaking, and then allowing to stand for 10 minutes.

In addition, the foam thickness and absorbance values at an added water quantity of 5 mL (on the horizontal axis of the graph) were 0.5 and 1.1 respectively.

The Low Moisture Enzyme Reaction (LMER) used in the present invention is a technique that is superior to the Para-Powder State Enzyme Reaction (PPSER) that has already been proposed by the inventors of the present invention, and the inventors of the present invention discovered the dynamics of enzyme reactions brought about by altering the quantity of water in the process of observing the reactions. This technique shows that “the world of low moisture” exists in enzyme reaction environments.

With regard to oil/fat raw materials, oils/fats include a variety of materials and components, of which characteristics are varied. Oils/fats able to be used in the present invention are not limited, and in order to produce food materials, it is desirable to use materials/components which can be procured in large quantities, are easy to handle, have a good flavor and are excellent in terms of health and physiological functionality. Examples of materials/components that meet these requirements include rapeseed oil, palm oil, soybean oil, rice oil, coconut oil, cooking oil, Resetta, oleic acid, linolic acid, EPA and DHA, but it is also possible to use an extremely broad range of materials/components, such as sesame oil, olive oil, safflower oil, sunflower oil, linseed oil, coconut oil and cocoa butter, which are plant-based oils, and liver oil, fish oil, whale oil, chicken oil, horse oil, lard, beef tallow, lard and butter, which are animal-based oils/fats. Oils/fats that are oily at room temperature can be used without further modification, and oils/fats in a sol-gel state can be incorporated without further modification, but by melting oils/fats in a sol-gel state by means of slight heating, such oils/fats can be used or mixed with other liquid oils/fats while molten. Such oils/fats can be dissolved in ethanol.

Essential oils and the like can also be used in the method of the present invention. Organic acid salts such as sodium acetate, potassium acetate, calcium acetate and sodium succinate, inorganic acid salts such as sodium sulfate and sodium phosphate, fatty acid salts, and esters having an R—COOX (X is a metal atom such as sodium, an ethyl group, or the like) structure, such as ethyl acetate, can be used, and materials obtained by blending oils/fats with other components can also be used.

### Test Example 2

In this test example, foam-forming properties and emulsion-forming properties achieved by means of a lipase reaction of an oil/fat or a polyhydric unsaturated fatty acid in isolation [oil/fat in isolation+water quantity (moisture content relative to dry weight of oil/fat 100 [d.b.%])] were investigated.

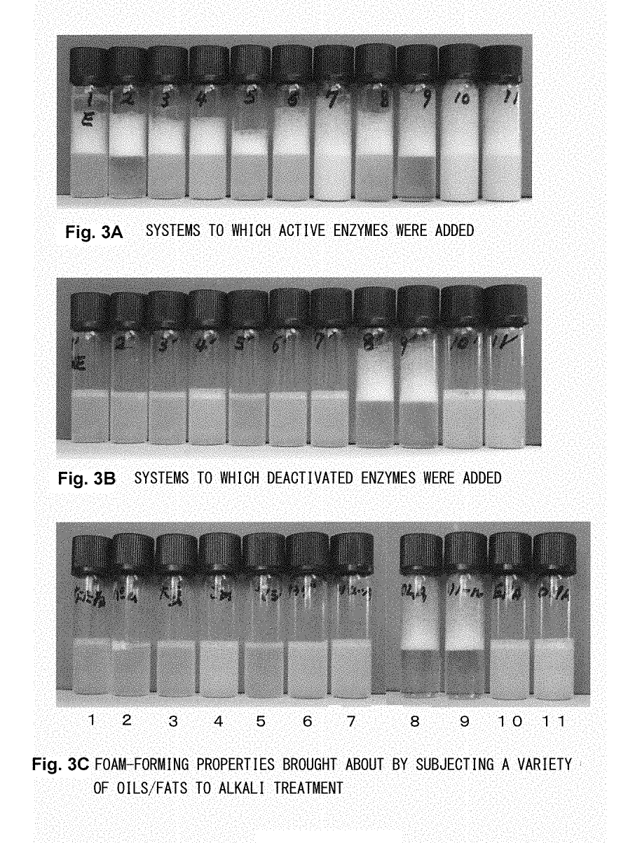

These results are shown in FIG. 3A to 3C. In FIG. 3A to 3C, 1 to 11 denotes the number of each test group, the type of oil/fat in each test group is as follows: 1. rapeseed oil, 2. palm oil, 3. soybean oil, 4. rice oil, 5. coconut oil, 6. cooking oil, 7. Resetta, 8. OLA, 9. linolic acid, 10. EPA, 11. DHA, are for 50 mg of oil/fat+50 μL of a 1% active enzyme solution or 50 mg of oil/fat+50 μL of a 1% deactivated enzyme solution, and show the appearance after reacting for 2 days at a temperature of 40° C. in a closed system, adding 170 μL of 1N NaOH, mixing by agitating, adding 5 mL of water, agitating by shaking, and then allowing to stand for 10 minutes.

FIG. 3A shows foam-forming properties in systems to which active enzymes were added, FIG. 3B shows foam-forming properties in systems to which deactivated enzymes were added, and FIG. 3C shows foam-forming properties brought about by subjecting a variety of oils/fats to alkali treatment by adding 170 μL of 1N NaOH.

Moreover, emulsion-forming properties are recorded as emulsion forming values, and the level thereof is displayed as −, ±, +, ++ or +++ (none, slight, low, medium or high). Absorbance: Relationship to emulsion-forming properties are as follows. 0-2: −, 2-4: +, 4-6: ++, 6 or higher: +++, and 2− means 2 or more and −4 means less than 4. The quality of emulsion-forming properties is such that a higher value means superior emulsion-forming properties.

Foam-forming properties are recorded as foam forming values, and the level thereof is displayed as −, ±, +, ++ or +++ (none, slight, low, medium or high). Foam thickness (cm): Relationship to foam-forming properties are as follows. 0: −, 0-0.5: ±, 0.5-1 cm: +, 1-2 cm: ++, 2 cm or more: +++.

Moreover, the vial had a total length of 8 cm, with the lid part accounting for 2 cm and space inside the vial accounting for 4.5 cm (or 2.5 cm when 5 mL of a liquid phase was present inside the vial).

The foam-forming properties, emulsion-forming properties and pH of each oil/fat are listed below in order to facilitate comparison.

In Table 1, A shows foam-forming properties test nos. 1 to 11 and results thereof, B shows emulsion-forming properties test nos. 1 to 11 and results thereof, and C shows pH test nos. 1 to 11 and results thereof.

TABLE 1
A

| | Foam-forming properties test no. | | | | | | | | | | |
|---|---|---|---|---|---|---|---|---|---|---|---|
| | 1 | 2 | 3 | 4 | 5 | 6 | 7 | 8 | 9 | 10 | 11 |
| Active | +++ | +++ | +++ | +++ | ++ | +++ | +++ | +++ | +++ | +++ | +++ |
| enzyme added | | | | | | | | | | | |
| Deactivated | − | − | − | ± | − | − | ± | +++ | +++ | ± | ± |
| enzyme added | | | | | | | | | | | |

B

| | Emulsion-forming properties test no. | | | | | | | | | | |
|---|---|---|---|---|---|---|---|---|---|---|---|
| | 1 | 2 | 3 | 4 | 5 | 6 | 7 | 8 | 9 | 10 | 11 |
| Active | + | − | ++ | ++ | ++ | ++ | +++ | ++ | − | +++ | +++ |
| enzyme added | | | | | | | | | | | |
| Deactivated | + | + | + | ++ | + | ++ | ++ | − | − | ++ | ++ |
| enzyme added | | | | | | | | | | | |

C

| | pH test no. | | | | | | | | | | |
|---|---|---|---|---|---|---|---|---|---|---|---|
| | 1 | 2 | 3 | 4 | 5 | 6 | 7 | 8 | 9 | 10 | 11 |
| Active | 7 | 7 | 8 | 8 | 7 | 8 | 7 | 8 | 8 | 7 | 7 |
| enzyme added | | | | | | | | | | | |
| Deactivated | 11 | 11 | 11 | 11 | 11 | 11 | 11 | 7 | 11 | 11 | 11 |
| enzyme added | | | | | | | | | | | |

As shown in the drawings and table, systems to which active enzymes were added exhibited significantly higher foam-forming properties, and systems to which deactivated enzymes were added exhibited almost no foam-forming properties, with the exception of nos. 8 and 9. It should be noted that higher emulsion-forming properties were exhibited by systems 3, 5, 7, 8, 10 and 11 in cases where active enzymes were added, that system 2 exhibited higher emulsion-forming properties when a deactivated enzyme was added than when an active enzyme was added, and that no emulsion-forming properties were exhibited by some systems to which active enzymes were added.

With regard to pH, the pH was 7 to 8 in systems to which active enzymes were added, and this value was maintained even after adding a 1N NaOH solution. Among systems to which deactivated enzymes were added, only no. 8 (OLA) maintained a pH of 7, and all the other systems had a pH of 11 or higher even when a small quantity (10 μL) of a 1N NaOH solution was added, and the difference in activity according to the number of double bonds should be noted. The pH was 7 to 8 in test groups following active lipase reactions, and because the pH in these test groups did not change even after adding a certain quantity of an alkaline liquid, it is thought that components having buffering effects may be produced by lipase reactions.

Incidentally, when a system comprising 50 mg of rapeseed oil and 50 μL of a 1% deactivated enzyme solution was observed after treating for 3 hours at a temperature of 40° C. in a closed system, adding 170 μL of a 1N NaOH solution, mixing by agitating, adding 5 mL of water, agitating by shaking and then allowing to stand for 10 minutes, foam-forming properties were evaluated as ±, emulsion-forming properties were evaluated as −, and the pH was 11. In addition, when observations were carried out after collecting 50 mg of oil/fat, adding 5 mL of water, adding 170 μL of a 1N NaOH solution, mixing by agitating, and then allowing to stand at room temperature for 10 minutes, the results show that oleic acid and linolic acid exhibited foam-forming properties, but other oils/fats used did not exhibit foam-forming properties, as indicated below.

### Test Example 3

In this test example, foam-forming properties and emulsion-forming properties [sucrose+various oils/fats] were investigated when carrying out lipase reactions using only sucrose as the carbohydrate and altering the type of oil/fat.

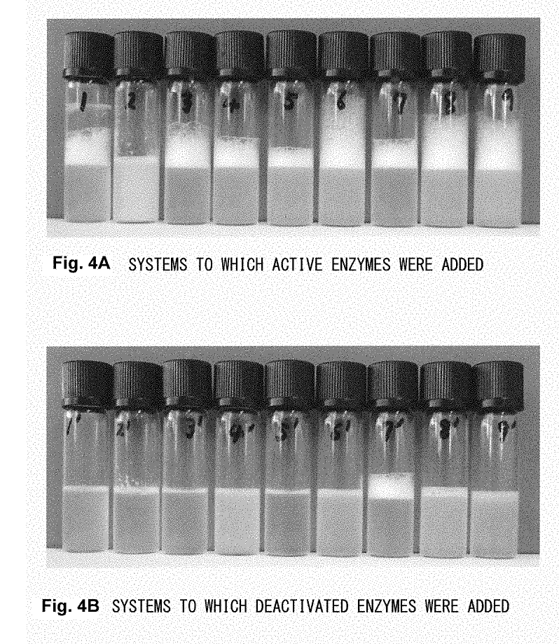

Investigations were carried out using Suc+a variety of oils/fats±LE (+LE means an active enzyme was added, and −LE means a deactivated enzyme was added) as a reaction system by using only sucrose as the carbohydrate and altering the type of oil/fat. Foam-forming properties, emulsion-forming properties and pH values were checked after carrying out a reaction for 3 hours at a temperature of 40° C. in a closed system, adding 170 μL of a 1N NaOH solution, adding 5 mL of water, agitating by shaking, and then allowing to stand for 10 minutes. Among test nos. 1 to 9, the types of oil/fat were as follows: 1. rapeseed oil, 2. palm oil, 3. soybean oil, 4. rice oil, 5. coconut oil, 6. Resetta, 7. OLA, 8. EPA, 9. DHA. These results are shown in FIGS. 4A and 4B.

FIG. 4A shows systems to which active enzymes were added, and FIG. 4B shows systems to which deactivated enzymes were added.

In Table 2, A shows foam-forming properties test nos. 1 to 9 and results thereof, B shows emulsion-forming properties test nos. 1 to 9 and results thereof, and C shows pH test nos. 1 to 9 and results thereof.

TABLE 2
A

| | Foam-forming properties test no. | | | | | | | | |
|---|---|---|---|---|---|---|---|---|---|
| | 1 | 2 | 3 | 4 | 5 | 6 | 7 | 8 | 9 |
| Active | +++ | ± | ++ | ++ | + | +++ | ++ | +++ | +++ |
| enzyme added | | | | | | | | | |
| Deactivated | − | − | − | − | − | − | ++ | + | − |
| enzyme added | | | | | | | | | |

B

| | Emulsion-forming properties test no. | | | | | | | | |
|---|---|---|---|---|---|---|---|---|---|
| | 1 | 2 | 3 | 4 | 5 | 6 | 7 | 8 | 9 |
| Active | + | +++ | + | + | ++ | ++ | + | ++ | +++ |
| enzyme added | | | | | | | | | |
| Deactivated | + | + | + | ++ | + | ++ | + | ++ | ++ |
| enzyme added | | | | | | | | | |

C

| | pH test no. | | | | | | | | |
|---|---|---|---|---|---|---|---|---|---|
| | 1 | 2 | 3 | 4 | 5 | 6 | 7 | 8 | 9 |
| Active | 7 | 7 | 8 | 8 | 7 | 8 | 7 | 8 | 8 |
| enzyme added | | | | | | | | | |
| Deactivated | 11 | 11 | 11 | 11 | 11 | 11 | 7 | 11 | 11 |
| enzyme added | | | | | | | | | |

Even in cases where Suc was added to an oil/fat and a lipase was caused to act on the mixture, the mixture exhibited foam-forming properties. In terms of emulsion-forming properties, it was understood that when Suc was added, there were test groups that differed from groups to which Suc was not added and groups comprising only oils/fats, but that these groups were similar overall to test groups comprising only oils/fats. In particular, it should be noted that palm oil to which Suc was added exhibited low foam-forming properties, but high emulsion-forming properties, and that EPA and DHA exhibited low emulsion-forming properties.

### Test Example 4

In this test example, foam-forming properties and emulsion-forming properties [sorbitol+a variety of oils/fats] were investigated when replacing the carbohydrate with sorbitol and carrying out lipase reactions on a variety of oils/fats.

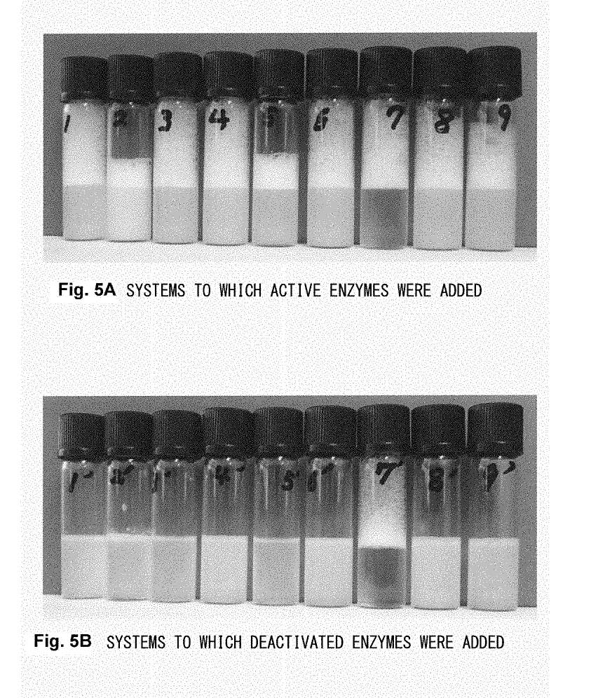

In Test Example 3, it was investigated whether a lipase reaction was facilitated when using Sor as a carbohydrate. These results are shown in FIGS. 5A and 5B.

FIG. 5A shows systems to which active enzymes were added, and FIG. 5B shows systems to which deactivated enzymes were added.

In Table 3, A to C show foam-forming properties test nos. 1 to 9 and results thereof, B shows emulsion-forming properties test nos. 1 to 9 and results thereof, and C shows pH test nos. 1 to 9 and results thereof.

In view of these results, it was found that a reaction progressed better. In particular, palm oil exhibited high emulsion-forming properties, and foam-forming properties were exhibited by all the oils/fats.

TABLE 3
A

| | Foam-forming properties test no. | | | | | | | | |
|---|---|---|---|---|---|---|---|---|---|
| | 1 | 2 | 3 | 4 | 5 | 6 | 7 | 8 | 9 |
| Active | +++ | ++ | +++ | +++ | ++ | +++ | +++ | +++ | +++ |
| enzyme added | | | | | | | | | |
| Deactivated | − | ± | − | − | − | − | +++ | − | − |
| enzyme added | | | | | | | | | |

B

| | Emulsion-forming properties test no. | | | | | | | | |
|---|---|---|---|---|---|---|---|---|---|
| | 1 | 2 | 3 | 4 | 5 | 6 | 7 | 8 | 9 |
| Active | + | +++ | + | ++ | + | ++ | − | ++ | + |
| enzyme added | | | | | | | | | |
| Deactivated | + | + | + | ++ | + | ++ | − | ++ | + |
| enzyme added | | | | | | | | | |

C

| | pH test no. | | | | | | | | |
|---|---|---|---|---|---|---|---|---|---|
| | 1 | 2 | 3 | 4 | 5 | 6 | 7 | 8 | 9 |
| Active | 8 | 7 | 8 | 8 | 7 | 8 | 7 | 8 | 8 |
| enzyme added | | | | | | | | | |
| Deactivated | 11 | 11 | 11 | 11 | 11 | 11 | 7 | 11 | 11 |
| enzyme added | | | | | | | | | |

### Test Example 5

In this test example, foam-forming properties and emulsion-forming properties [rapeseed oil+a variety of carbohydrates] were investigated when using only rapeseed oil as the oil/fat and carrying out a lipase reaction when adding a variety of carbohydrates.

Because it was found that foam-forming properties and emulsion-forming properties varied according to the type of carbohydrate, tests were carried out by using only rapeseed oil as the oil/fat, selecting the type of carbohydrate, and investigating how foam-forming properties and emulsion-forming properties varied in systems comprising rapeseed oil+a variety of carbohydrates±LE in order to investigate the effects achieved by a variety of carbohydrates.

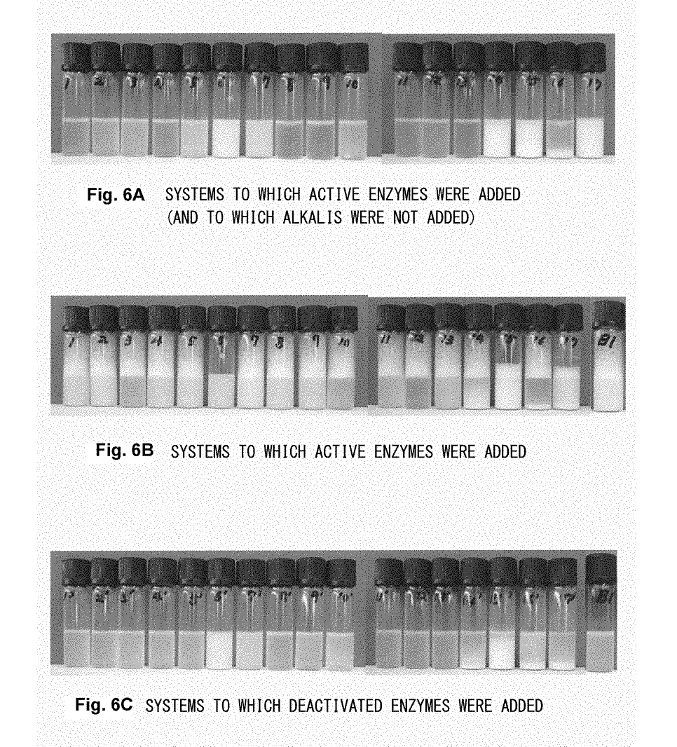

After weighing out 250 mg of a carbohydrate, adding 50 mg of rapeseed oil, adding 50 μL of 1% LE or 50 μL of deactivated 1% LE, mixing by agitating, reacting for 3 hours at a temperature of 40° C. in a closed system, adding 5 mL of water, adding 170 μL of a 1N NaOH solution, agitating by shaking and then allowing to stand for 10 minutes, the pH was checked using a pH test paper and foam-forming properties and emulsion-forming properties were observed. FIGS. 6A to 6C show results for systems to which active enzymes were added (and to which an alkali was not added), systems to which active enzymes were added and systems to which deactivated enzymes were added.

Moreover, in FIGS. 6A to 6C and FIGS. 7, 1 to 17 denote test group numbers, and the types and abbreviations of carbohydrates selected and used in the test groups being as follows: 1. xylose (Xyl), 2. fructose (Fru), 3. acetylglucosamine (GNAc), 4. glucose (Glc), 5. maltose monohydrate (G2.H₂O), 6. soluble starch (Gn), 7. sorbitol (Sor), 8. erythritol (Er), 9. xylitol (XylOH), 10. mannitol (ManOH), 11. sucrose (Suc), 12. lactose (Lac), 13. trehalose (Tr), 14. corn starch (CS), 15. glutinous rice starch (WRS), 16. rice flour (top-grade rice flour made from non-glutinous rice) (RP), 17. cellulose (Cel).

B1 shows the appearance after reacting 50 mg of rapeseed oil and 50 μL of LE for 3 hours at a temperature of 40° C. in a closed system, heat treating for 10 minutes in a boiling water bath, adding 5 mL of water, agitating by shaking, adding 170 μL of 1N NaOH, agitating by shaking, and then allowing to stand at room temperature for 10 minutes.

B1′ shows the appearance after reacting 50 mg of rapeseed oil and 50 μL of deactivated LE for 3 hours at a temperature of 40° C. in a closed system, heat treating for 10 minutes in a boiling water bath, adding 5 mL of water, agitating by shaking, adding 170 μL of 1N NaOH, agitating by shaking, and then allowing to stand at room temperature for 10 minutes.

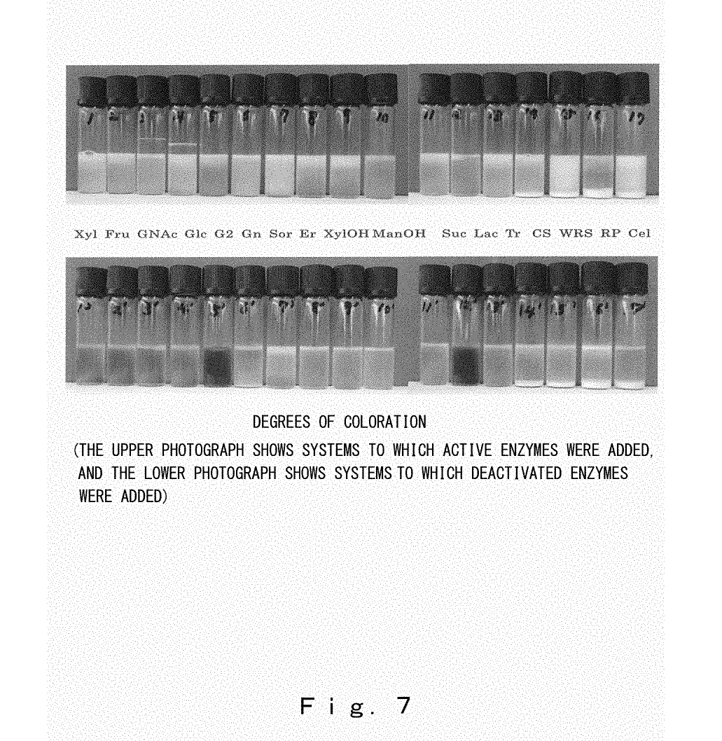

FIG. 7 shows degrees of coloration (the upper photograph shows systems to which active enzymes were added, and the lower photograph shows systems to which deactivated enzymes were added).

Table 4 shows changes in a variety of functions in systems in which a variety of carbohydrates were added.

TABLE 4
Changes in a variety of functions in systems in which a variety of carbohydrates were added

| | Test no. | | | | | | | | | | | | | | | | | |
|---|---|---|---|---|---|---|---|---|---|---|---|---|---|---|---|---|---|---|
| | 1 | 2 | 3 | 4 | 5 | 6 | 7 | 8 | 9 | 10 | 11 | 12 | 13 | 14 | 15 | 16 | 17 | Bnk |
| Foam-forming | +++ | +++ | +++ | +++ | +++ | − | +++ | +++ | +++ | +++ | +++ | +++ | +++ | +++ | + | +++ | + | +++ |
| properties | | | | | | | | | | | | | | | | | | |
| (active enzyme | | | | | | | | | | | | | | | | | | |
| added) | | | | | | | | | | | | | | | | | | |
| Foam-forming | All− | | | | | | | | | | | | | | | | | |
| properties | | | | | | | | | | | | | | | | | | |
| (deactivated | | | | | | | | | | | | | | | | | | |
| enzyme added) | | | | | | | | | | | | | | | | | | |
| Emulsion-forming | +++ | +++ | − | ++ | +++ | +++ | +++ | ++ | ++ | ± | + | − | + | ++ | +++ | − | ++ | ++ |
| properties | | | | | | | | | | | | | | | | | | |
| (active enzyme | | | | | | | | | | | | | | | | | | |
| added) | | | | | | | | | | | | | | | | | | |
| Foam-forming | + | + | + | + | + | +++ | ++ | ± | + | + | − | − | − | + | ++ | + | + | + |
| properties | | | | | | | | | | | | | | | | | | |
| (deactivated | | | | | | | | | | | | | | | | | | |
| enzyme added) | | | | | | | | | | | | | | | | | | |
| pH (active | 8 | 8 | 8 | 8 | 9 | 11 | 9 | 8 | 11 | 8 | 8 | 8 | 8 | 9 | 8 | 11 | 8 | 11 |
| enzyme added) | | | | | | | | | | | | | | | | | | |
| pH (deactivated | All 11 | | | | | | | | | | | | | | | | | |
| enzyme added) | | | | | | | | | | | | | | | | | | |
| Coloration | − | ++ | − | − | +++ | + | − | − | − | − | − | ± | − | − | − | − | − | |
| (active enzyme | | | | | | | | | | | | | | | | | | |
| added) | | | | | | | | | | | | | | | | | | |
| Coloration | ++ | ++ | ++ | ++ | +++ | + | − | − | − | − | − | +++ | − | − | − | − | − | |
| (deactivated | | | | | | | | | | | | | | | | | | |
| enzyme added) | | | | | | | | | | | | | | | | | | |

As shown in FIG. 6A, foam-forming properties were not exhibited if an alkali was not added to a system to which an active enzyme was added, and nos. 6, 14, 15 and 17 exhibited high emulsion-forming properties, but it was understood that high emulsion-forming properties were generally exhibited by systems to which an alkali was added, as shown in FIG. 6B.

The degree of coloration was observed after hermetically sealing the vial and allowing the vial to stand at room temperature for 4 weeks, and maltose and lactose in particular underwent a high degree of coloration, and xylose, fructose, acetylglucosamine, glucose and soluble starch underwent some degree of coloration. In general, systems to which a deactivated enzyme was added underwent a high degree of coloration, and in cases where a lipase reaction was carried out after adding a carbohydrate, stability was increased and emulsion-forming properties were stably maintained. Therefore, because foam-forming properties and emulsion-forming properties were varied in systems in which oils/fats were combined with a carbohydrate, such combinations should be selected as appropriate according to the intended use of the product.

Other carbohydrate components and materials can be used as carbohydrate raw materials in the present invention, and a variety of new materials can be produced by using the method of the present invention on, for example, agricultural produce such as cereal grains, beans, fruits, leafy vegetables, stem vegetables, flower vegetables, potatoes, tea, mushrooms, algae and microalgae; and hydrophobic materials and components obtained from waste products and by-products discharged when processing such agricultural produce. Specifically, functional materials having foam-forming properties and emulsion foaming properties can be produced using the method of the present invention by further adding an oil/fat to a residue obtained by adding an organic solvent solution such as hexane, acetone or an alcohol to waste products and by-products, such as rice bran, wheat gluten cake, skin or seeds discharged when processing rice, barley, yuzu (a type of citrus fruit), mandarin oranges, pineapples, herbs, tea, and the like, extracting, and then distilling off (recovering) the organic solvent. Therefore, the method of the present invention can contribute to a reduction in the burden on the environment by using agricultural produce and food raw materials to the greatest possible extent, and final residues can be returned to farmland and used to improve soil quality. Alcohols act together with carbohydrate-related enzymes and are partially converted into alcohol glucosides, and it is possible to obtain food materials containing these.

### Test Example 6

In this test example, foam-forming properties and emulsion-forming properties [rapeseed oil+sorbitol+a variety of alkaline materials] were investigated when using only rapeseed oil as the oil/fat, carrying out a lipase reaction and adding a variety of alkaline materials and components.

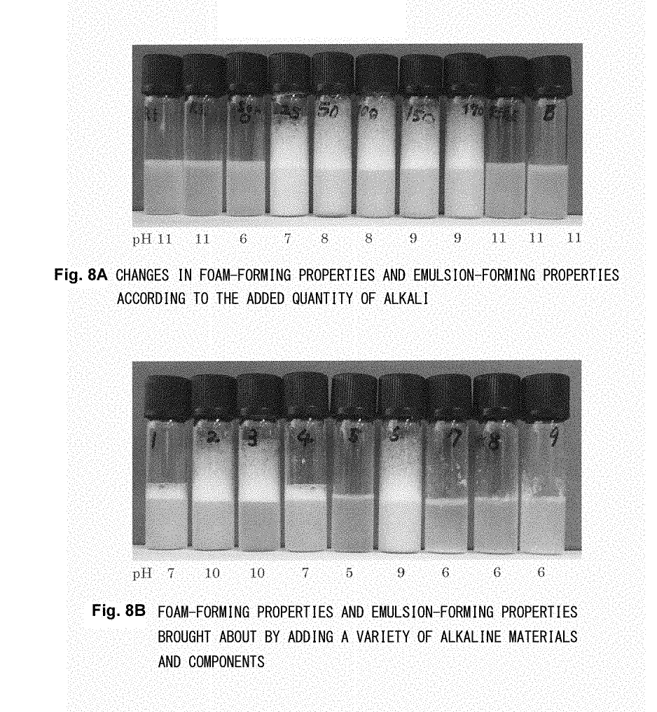

First, 8 specimens were prepared by reacting 50 mg of rapeseed oil, 250 mg of Sor and 50 μL of LE for 3 hours at a temperature of 40° C. in a closed system and heat treating for 10 minutes in a boiling water bath. Observations were carried out after adding 5 mL of water, agitating by shaking, adding 0 μL, 25 μL, 50 μL, 100 μL, 150 μL or 170 μL of 1N NaOH, agitating by shaking, and allowing to stand at room temperature for 10 minutes. These results are shown in FIGS. 8A and 8B.

In FIG. 8B, numbers 1 to 9 denote test groups numbers, and alkaline materials and components used in the test are as follows: 1. NaHCO₃, 2. Na₂CO₃, 3. K₂CO₃, 4. K₂HPO₄, 5. KH₂PO₄, 6. soda ash, 7. calcium carbonate (seashell Ca), 8. CalMag-S, 9. calcium phosphate (Fired Bonical).

In the FIGS. 8A and 8B, A shows changes in foam-forming properties and emulsion-forming properties according to the added quantity of alkali, and B shows foam-forming properties and emulsion-forming properties brought about by adding a variety of alkaline materials and components.

“Deactivated” is a system obtained by processing under similar conditions to a reaction between 250 mg of Sor, 50 mg of rapeseed oil and 50 μL of deactivated LE.

B shows observations after allowing 50 mg of rapeseed oil to stand at room temperature for 3 hours, then simultaneously adding 170 μL of 1N NaOH and 5 mL of water, and then agitating.

At an added alkali quantity of 5 μL and 10 μL, foam-forming properties and emulsion-forming properties were exhibited, and the pH was approximately 7.

In systems comprising 50 mg of rapeseed oil and 50 μL of LE, broadly similar results were obtained when carrying out similar reactions.

The diagrams show the results of observations after reacting 50 mg of rapeseed oil and 50 μL of LE for 3 hours at a temperature of 40° C. in a closed system, carrying out deactivation treatment for 10 minutes in a boiling water bath, adding 10 mg (10 to 11 mg) of an alkaline material, adding 5 mL of water while agitating, agitating by shaking, and then allowing to stand for 10 minutes at room temperature.

Because foam-forming properties and emulsion-forming properties varied according to the type of material/component, as shown in the diagrams, the type of material/component should be selected according to need.

As an explanation of LMER materials obtained using the production method of the present invention, these materials are produced by adding a small quantity of water to an oil/fat and then causing a lipase to act on the obtained mixture, the oil/fat is converted into a soft paste as a result of the reaction between the oil/fat and the water, and a product obtained by filling a bottle or can with this soft paste can be used as an oil/fat LMER precursor material together with a variety of materials when producing confectionery or beverages. An alkaline material or component is also used in cases where foam-forming properties or emulsion-forming properties are required. Products produced by adding a carbohydrate to an oil/fat can be used as carbohydrate-oil/fat LMER precursor materials or carbohydrate-oil/fat LMER materials when producing a variety of foods. Such products have abroad scope of use for producing noodles, bread, rice, confectionery and beverages.

In order to ameliorate the characteristic odor of oils/fats, a lipase reaction is carried out after drying an ethanol solution extract of tea or herbal tea and mixing this dried extract with oils/fats. Furthermore, extracts of dried powders of heart's ease, plant-derived sweet materials and essential oils can also be used.

The LMER method per se can also be used to produce a desired food by carrying out a lipase reaction after mixing an oil/fat with a material other than an oil/fat or directly adding an appropriate quantity of water to a raw material containing an oil/fat. It is possible to alter the functionality of a material or component. For example, by causing a lipase to act in a low moisture state when producing a fermented food, bread, noodles or rice, improvements in product quality and taste quality can be expected. It can be reasoned that low moisture reactions of amino acids, peptides and protein-related enzymes can also be carried out by developing the method of the present invention. Broadly speaking, low moisture reactions are involved in facilitating or suppressing functions of physiologically functional components (including enzymes), and it is expected that many applications will be found for low moisture reactions.

By combining an enzyme, or a combination of two or more types thereof, with a substrate, or a combination of two or more types thereof, it is possible to produce a wide variety of food materials, ingredients and products. Furthermore, by appropriately combining physiologically active substances, such as carbohydrates, proteins, lipids, nucleic acids and other polyphenols, with a variety of related enzymes, it is predicted that a wide variety of complex materials (hybrid materials) can be produced by means of low moisture reactions.

The present invention will now be explained in greater detail through the use of working examples, but is in no way limited to the working examples given below.

### Working Example 1

2 g of rapeseed oil and 2 mL of a 0.1% aqueous enzyme solution of lipase AY “Amano” 30SD manufactured by Amano Enzyme Inc. were placed in a bottle equipped with a lid and thoroughly agitated, after which the bottle was sealed with the lid and the contents of the bottle were allowed to stand and react at a temperature of 40° C. for 18 hours. At this point, the moisture content relative to the dry weight of the oil/fat was 100 [d.b.%]. Following the reaction, the reaction mixture was, in this condition, subjected to enzyme deactivation heat treatment by being heated for 30 minutes at a temperature of 105° C., thereby obtaining a greasy product.

Similar greasy products were obtained in the same way, except that palm oil, soybean oil, rice oil, coconut oil, cooking oil, Resetta, oleic acid, linolic acid, EPA or DHA was used instead of rapeseed oil. In addition, similar greasy products were obtained in the same way by using olive oil or sesame oil as the oil/fat. (Oil/fat-lipase reaction product, precursor material)

### Working Example 2

A similar greasy product was obtained in the same way as in Working Example 1 by using a mixed oil/fat containing equal quantities of rapeseed oil and coconut oil and a mixed oil/fat containing equal quantities of rice oil and palm oil.

### Working Example 3

EPA and DHA products having no unpleasant odor were obtained in the same way as in Working Example 1, except that EPA or DHA was used as the oil/fat and treated by being placed in a product obtained by extracting green tea with 50% ethanol and then drying.

Moreover, the product obtained by extracting green tea with 50% ethanol and then drying was prepared by adding 10 mL of a 50% ethanol solution to 2 g of green tea, extracting for 18 hours at room temperature with occasional agitation, withdrawing 5 mL of the supernatant liquid, and drying this supernatant liquid at a temperature of 105° C.

### Working Example 4

2 g of rapeseed oil, 10 g of sorbitol and 2 mL of a 0.1% aqueous enzyme solution of lipase AY “Amano” 30SD manufactured by Amano Enzyme Inc. were placed in a bottle equipped with a lid and thoroughly agitated, after which the bottle was sealed with the lid and the contents of the bottle were allowed to stand and react at a temperature of 40° C. for 18 hours. At this point, the moisture content relative to the dry weight of the oil/fat was 100 [d.b.%]. Following the reaction, the lid was opened and the reaction mixture was subjected to enzyme deactivation heat treatment by being heated for 30 minutes at a temperature of 105° C., thereby obtaining a white powdered product. (Carbohydrate-oil/fat LMER precursor material)

Carbohydrates able to be used in the present invention are not particularly limited, and products produced using, for example, erythritol, xylitol or glutinous rice starch exhibit excellent emulsion-forming properties and high stability.

### Working Example 5

2 g of rapeseed oil, 1 g of sucrose and 1 g of glutinous rice starch were placed in a bottle equipped with a lid, 2 mL of a 0.1% aqueous enzyme solution of lipase AY “Amano” 30SD manufactured by Amano Enzyme Inc. was added to the bottle and thoroughly agitated, after which the bottle was sealed with the lid and the contents of the bottle were allowed to stand and react at a temperature of 40° C. for 18 hours. At this point, the moisture content relative to the dry weight of the oil/fat was 100 [d.b.%]. Following the reaction, the lid was opened and the reaction mixture was subjected to enzyme deactivation heat treatment by being heated for 30 minutes at a temperature of 105° C., thereby obtaining a white powdered product. (Carbohydrate-oil/fat LMER precursor material)

### Working Example 6

A product having excellent solubility and sweetness and good palatability was obtained in the same way as in Working Example 5, except that 1 mL of the 0.1% aqueous enzyme solution of lipase AY “Amano” 30SD manufactured by Amano Enzyme Inc. and 1 mL of a 0.1% aqueous enzyme solution of α-amylase (Kleistase L1) manufactured by Amano Enzyme Inc. were used instead of 2 mL of the 0.1% aqueous enzyme solution of lipase AY “Amano” 30SD.

By adding 0.5 mL of the 0.1% aqueous enzyme solution of α-amylase (Kleistase L1), 0.5 mL of a 0.1% aqueous enzyme solution of glucoamylase (Gluczyme AF6) and 1 mL of a 0.1% aqueous lipase solution, it was possible to further improve solubility.

By adding 1 mL of a 0.1% aqueous enzyme solution of cyclodextrin forming enzyme (Konchizyme-CGTase manufactured by Amano Enzyme Inc.) instead of [0.5 mL of the 0.1% aqueous enzyme solution of α-amylase (Kleistase L1) and 0.5 mL of a 0.1% aqueous enzyme solution of glucoamylase (Gluczyme AF6)], it was possible to improve stability.

### Working Example 7

2 g of rapeseed oil, 10 g of cellulose and 2 mL of a 0.1% aqueous enzyme solution of lipase AY “Amano” 30SD manufactured by Amano Enzyme Inc. were placed in a bottle equipped with a lid and thoroughly agitated, after which the bottle was sealed with the lid and the contents of the bottle were allowed to stand and react at a temperature of 40° C. for 18 hours. At this point, the moisture content relative to the dry weight of the oil/fat was 100 [d.b.%]. Following the reaction, the lid was opened and the reaction mixture was subjected to enzyme deactivation heat treatment by being heated for 30 minutes at a temperature of 105° C., thereby obtaining a white powdered product. (Carbohydrate-oil/fat LMER precursor material)

### Working Example 8

Instead of 2 mL of the 0.1% aqueous enzyme solution of lipase AY “Amano” 30SD manufactured by Amano Enzyme Inc., 1 mL of the 0.1% aqueous enzyme solution of lipase AY “Amano” 30SD and 1 mL of a 1% aqueous enzyme solution of a cellulase manufactured by Nagase ChemteX Corporation (Cellulase SS) were placed in a bottle equipped with a lid and thoroughly agitated, after which the bottle was sealed with the lid and the contents of the bottle were allowed to stand and react at a temperature of 40° C. for 18 hours. At this point, the moisture content relative to the dry weight of the oil/fat was 100 [d.b.%]. Following the reaction, the lid was opened and the reaction mixture was subjected to enzyme deactivation heat treatment by being heated for 30 minutes at a temperature of 105° C., thereby obtaining a white powdered product.

### Working Example 9

A product having excellent dispersibility/solubility was obtained in the same way as in Working Example 7, except that 0.5 mL of a 1% aqueous enzyme solution of the cellulase (Cellulase SS) and 0.5 mL of a hemicellulase manufactured by HBI Enzymes Inc. (Cellulosin HC) were used instead of 1 mL of a 1% aqueous enzyme solution of a cellulase manufactured by Nagase ChemteX Corporation (Cellulase SS).

Corresponding to claims 3 and 5

### Working Example 10

By adding 250 μL of a 1N NaOH solution to the products of Working Examples 1 to 3 and agitating, it was possible to obtain a product that exhibited foam-forming properties and emulsion-forming properties when dispersed/dissolved in water. However, when palm oil in isolation was subjected to a lipase reaction and an alkali was then added, a product having emulsion-forming properties was not obtained. (Oil/fat LMER precursor material)

### Working Example 11

By adding 10 mg of soda ash to the products of Working Examples 4 to 9 and agitating, it was possible to obtain a product that exhibited foam-forming properties and emulsion-forming properties when dispersed/dissolved in water. (Carbohydrate-oil/fat LMER material)

### Working Example 12

### Applied Example 1 as a Food Material

### Use in Beverage-Milk Beverage, Tea Beverage and Coffee Beverage

A food material was obtained by adding 10 mg of soda ash to a product obtained using Resetta instead of rapeseed oil in the manner described in Working Example 1, mixing and agitating, adding 20 mL of a cows milk raw material, a tea raw material or a coffee raw material, and then agitating by shaking. The products were homogeneous dispersions and maintained emulsion-forming properties even after being allowed to stand for 4 weeks at room temperature.

Health-oriented products were also obtained in cases where EPA or DHA was used instead of Resetta.

### Working Example 13

### Applied Example 2 as a Food Material

In order to be used in confectionery production, 10 mg of soda ash was added to the product obtained in Working Example 4 and mixed by means of agitation, after which 12 g of low gluten flour and 24 mL of a 1% saline solution were added and mixed by means of agitation. A cookie type confectionery was produced by dividing the obtained mixture into 2 portions and baking for 12 minutes in an oven at a temperature of 180° C.

An udon product having a good flavor was obtained by adding 12 g of low gluten flour and 6 mL of a 1% saline solution to 12 g of a carbohydrate-oil/fat LMER material produced using glutinous rice starch-rice oil-soda ash, mixing by means of agitation, molding, and boiling for 10 minutes.

### INDUSTRIAL APPLICABILITY

As explained in detail above, the present invention can produce an oil/fat-lipase reaction product of a precursor material (known as an oil/fat LMER precursor material) having latent foam-forming properties and emulsion-forming properties by causing a lipase to act on an oil/fat in a low moisture state, that is, in a state whereby the moisture content relative to the dry weight of the oil/fat is 4 to 400 [d.b.%] (the added quantity of water is 2 to 200 μL relative to 50 mg of the oil/fat), and can, by combining a carbohydrate with this oil/fat-lipase reaction product of a precursor material, produce a precursor carbohydrate-oil/fat-lipase reaction product (known as a carbohydrate-oil/fat LMER precursor material) having latent foam-forming properties and emulsion-forming properties.

In addition, by adding an alkaline component or material to the reaction product mentioned above and then foaming, emulsifying or powdering, the present invention can use the entire reaction product as a food material (known as an oil/fat LMER material or carbohydrate-oil/fat LMER material), with these products able to be widely used to produce foods, and by adding the LMER material to, for example, wheat flour, it is possible to contribute an expansion in the scope of use of brown rice flour and polished rice flour, such as producing confectionery or forming a slurry or paste by adding a small quantity of water.

In the present invention, as the most drastic usage method, it is possible to simply add a small quantity of a lipase solution directly to an oil/fat liquid, and mix by means of agitation so as to bring about a reaction, but by also adding a carbohydrate such as wheat flour or rice flour to the reaction mixture and bringing about a reaction, it is possible to use the present invention to produce bread, noodles or fermented foods, and by adding an alkaline material or component when necessary, it is possible to impart foam-forming properties or emulsion-forming properties. The LMER method according to the present invention is not limited to use in producing products such as those mentioned above, and because a buffering action is also observed, the present invention possesses industrial applicability, such as stably treating and coating functional groups in a variety of materials or components, for example replacing characteristics that have an adverse effect on health and lessening adverse reactions to drugs.

What is claimed is:

1. A method for producing an oil/fat-lipase reaction product of a precursor material having latent foam-forming properties and emulsion-forming properties by reacting an oil/fat or a combination of two or more types thereof with an enzyme lipase in a low moisture state in the presence of a lipase,
the method comprising carrying out a lipase reaction in which a moisture content relative to a dry weight of the oil/fat is 4 to 400 [d.b.%] (an added quantity of water is 2 to 200 μL relative to 50 mg of the oil/fat) as the enzyme reaction in a low moisture state.

2. A method for producing a carbohydrate-oil/fat-lipase reaction product of a precursor material having latent foam-forming properties and emulsion-forming properties by adding a carbohydrate to an oil/fat or a combination of two or more types thereof and carrying out an enzyme reaction in a low moisture state in the presence of a lipase,
the method comprising carrying out a lipase reaction in which a moisture content relative to a dry weight of the oil/fat is 4 to 400 [d.b.%] (an added quantity of water is 2 to 200 μL relative to 50 mg of the oil/fat) as the enzyme reaction in a low moisture state.

3. A method for producing an oil/fat-lipase reaction product or carbohydrate-oil/fat-lipase reaction product having latent foam-forming properties and emulsion-forming properties, the method comprising adding an alkaline component or raw material to the oil/fat-lipase reaction product or carbohydrate-oil/fat-lipase reaction product of a precursor material having latent foam-forming properties and emulsion-forming properties obtained in claim 1, and foaming, emulsifying or powdering, thereby using the entire reaction product as a food material.

4. The method according to claim 1, wherein rapeseed oil, soy bean oil, palm oil, coconut oil, cooking oil, Resetta, corn oil, safflower oil, olive oil, sesame oil, sunflower oil, rice oil or linseed oil, which are plant-based oils/fats; fish oil, whale oil, horse oil, lard or butter, which are animal-based oils/fats; or DHA, EPA, arachidonic acid, oleic acid, linolic acid or linolenic acid, which are fatty acids, is used as the oil/fat.

5. The method according to claim 2, wherein xylose, fructose, acetylglucosamine, glucose, maltose, soluble starch, sorbitol, erythritol, xylitol, mannitol, sucrose, lactose, trehalose, corn starch, rice starch, rice flour (top-grade rice flour) or cellulose is used as the carbohydrate.

6. The method according to claim 3, wherein sodium hydroxide, potassium hydroxide, sodium carbonate, sodium hydrogen carbonate, potassium carbonate, potassium hydrogen carbonate, sodium phosphate, sodium hydrogen phosphate, soda ash, calcium carbonate (seashell calcium), CalMag-S, calcium phosphate (Fired Bonical) or an ash extraction liquid is used as the alkaline component or raw material.

7. The method according to claim 2, wherein grain flour or starch is used as the carbohydrate and an α-amylase and/or a glucosidase is also used.

8. The method according to claim 7, wherein the grain flour is rice flour, white rice bran or wheat flour while the starch is waxy potato starch, sweet potato starch, tapioca starch, non-glutinous rice starch, glutinous rice starch, corn starch, glutinous corn starch, wheat starch or sago starch.

9. The method according to claim 5, wherein cellulose is used as the carbohydrate and a cellulase is also used.

10. The method according to claim 3, wherein an ash extraction liquid is added as an alkaline material.

11. A method for producing an oil/fat-lipase reaction product or carbohydrate-oil/fat-lipase reaction product having latent foam-forming properties and emulsion-forming properties, the method comprising adding an alkaline component or raw material to the oil/fat-lipase reaction product or carbohydrate-oil/fat-lipase reaction product of a precursor material having latent foam-forming properties and emulsion-forming properties obtained in claim 2, and foaming, emulsifying or powdering, thereby using the entire reaction product as a food material.

12. The method according to claim 2, wherein rapeseed oil, soy bean oil, palm oil, coconut oil, cooking oil, Resetta, corn oil, safflower oil, olive oil, sesame oil, sunflower oil, rice oil or linseed oil, which are plant-based oils/fats; fish oil, whale oil, horse oil, lard or butter, which are animal-based oils/fats; or DHA, EPA, arachidonic acid, oleic acid, linolic acid or linolenic acid, which are fatty acids, is used as the oil/fat.

13. The method according to claim 11, wherein sodium hydroxide, potassium hydroxide, sodium carbonate, sodium hydrogen carbonate, potassium carbonate, potassium hydrogen carbonate, sodium phosphate, sodium hydrogen phosphate, soda ash, calcium carbonate (seashell calcium), CalMag-S, calcium phosphate (Fired Bonical) or an ash extraction liquid is used as the alkaline component or raw material.

14. The method according to claim 11, wherein an ash extraction liquid is added as an alkaline material.
